(19) United States
(12) Patent Application Publication
(10) Pub. No.: US 2007/0299047 A1
(43) Pub. Date: Dec. 27, 2007

(54) Combination of Organic Compounds

(76) Inventors: William Maher, Richboro, PA (US); Michele Mercuri, Scotch Plains, NJ (US); Meenakshi Nevatia, Bottminger (CH); Hong Chen, Newton, MA (US); Pei-Ran Wang, Cambridge, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/574,994

(22) PCT Filed: Sep. 8, 2005
(86) PCT No.: PCT/US05/32224

§ 371 Date: Mar. 9, 2007

Related U.S. Application Data
(60) Provisional application No. 60/608,310, filed on Sep. 9, 2004.
(60) Provisional application No. 60/609,577, filed on Sep. 14, 2004.
(60) Provisional application No. 60/689,738, filed on Jun. 10, 2005.

(51) Int. Cl. A61K 31/422; A61K 31/397; A61K 31/4365; A61K 31/52; A61P 1/00; A61P 11/00; A61P 17/00; A61P 25/26; A61P 25/28; A61P 29/02; A61P 35/00; A61P 43/00; A61P 9/00

(52) U.S. Cl. 514/210.02; 514/210.17; 514/263.22; 514/301; 514/374

(57) ABSTRACT

The present invention relates to a pharmaceutical composition, comprising a PPAR agonist, or pharmaceutically acceptable salts thereof, alone or in combination with at least one active ingredient selected from the group consisting of (i) HDL increasing compounds; (ii) anti-diabetics; (iii) an anti-hypertensive agent; (iv) cholesterol absorption modulator; (v) apo-A1 analogs and mimetics; (vi) renin inhibitors; (vii) thrombin inhibitors; (viii) aldosterone inhibitors; (ix) GLP-1 agonists; (x) glucagon receptor antagonists; (xi) cannabinoid receptor 1 antagonists; (xii) anti-obesity agents; and (xiii) inhibitors of platelet aggregation or, in each case, a pharmaceutically acceptable salt thereof; and optionally a pharmaceutically acceptable carrier.

The present invention relates to a pharmaceutical composition, comprising a PPAR agonist, or pharmaceutically acceptable salts thereof, alone or in combination with at least one active ingredient selected from the group consisting of
(i) HDL increasing compounds;
(ii) anti-diabetics;
(iii) an anti-hypertensive agent;
(iv) cholesterol absorption modulator;
(v) apo-A1 analogs and mimetics;
(vi) renin inhibitors;
(vii) thrombin inhibitors;
(viii) aldosterone inhibitors;
(ix) GLP-1 agonists;
(x) glucagon receptor antagonists;
(xi) cannabinoid receptor 1 antagonists;
(xii) anti-obesity agents; and
(xiii) inhibitors of platelet aggregation or, in each case, a pharmaceutically acceptable salt thereof;
   and optionally a pharmaceutically acceptable carrier.

PPAR agonists are meant to include but not be limited to selective PPAR alpha agonists, PPAR gamma agonists or PPAR delta agonists and dual alpha/gamma agonists and dual alpha/delta agonists.

Selective PPAR alpha agonists include compounds of the formula 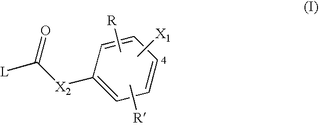
wherein L is a radical selected from the group consisting of: 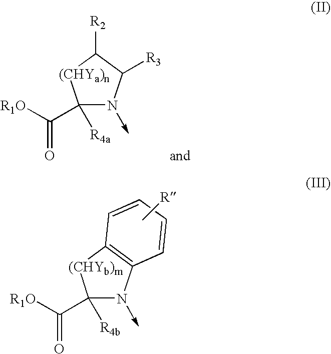
in which
R₁ is hydrogen, optionally substituted alkyl, aryl, heteroaryl, aralkyl or cycloalkyl;
R₂ is hydrogen, hydroxy, oxo, optionally substituted alkyl, aryl, aralkyl, alkoxy, aryloxy, aralkoxy, alkylthio, arylthio or aralkylthio;
R₃ is hydrogen; or
R₂ and R₃ combined are alkylene which together with the carbon atoms to which they are attached form a fused 5- to 7-membered ring; or
R₂ and R₃ combined are a bond between the carbon atoms to which they are attached;
n is zero or an integer of 1 or 2;
Yₐ is hydrogen; or
Yₐ and R₂ combined are a bond between the carbon atoms to which they are attached;
R₄ₐ is hydrogen; or
R₄ₐ and Yₐ combined are a bond between the carbon atoms to which they are attached;
R″ is hydrogen, optionally substituted alkyl, alkoxy or halogen;
m is an integer of 1 or 2;
Y_{b} is hydrogen;
R_{4b} is hydrogen; or
R_{4b} and Y_{b} combined are a bond between the carbon atoms to which they are attached;
R and R′ are independently hydrogen, halogen, optionally substituted alkyl, alkoxy, aralkyl or heteroaralkyl; or
R and R′ combined together with the carbon atoms to which they are attached form an optionally substituted fused 5- to 6-membered aromatic or heteroaromatic ring provided that R and R′ are attached to carbon atoms adjacent to each other; or
R—C and R′—C may independently be replaced by nitrogen;
X₁ is -Z-(CH₂)ₚ-Q-W wherein
   Z is a bond, O, S, S(O) or S(O)₂; or
   Z is —C(O)NR₅— in which
      R₅ is hydrogen, alkyl or aralkyl;
   p is an integer from 1 to 8;
   Q is a bond; or
   Q is —O(CH₂)ᵣ— or —S(CH₂)ᵣ in which
      r is zero or an integer from 1 to 8; or
   Q is —O(CH₂)₁₋₈O—, —S(CH₂)₁₋₈O—, —S(CH₂)₁₋₈S— or —C(O)—; or
   Q is —C(O)NR₆— in which
      R₆ is hydrogen, optionally substituted alkyl, cycloalkyl, aryl, heteroaryl, aralkyl or heteroaralkyl; or
   Q is —NR₇—, —NR₇C(O)—, —NR₇C(O)NR₈— or —NR₇C(O)O— in which
      R₇ is hydrogen, optionally substituted alkyl, cycloalkyl, aryl, heteroaryl, aralkyl or heteroaralkyl;
      R₈ is hydrogen, alkyl or aralkyl;
   W is cycloalkyl, aryl, heterocyclyl, aralkyl or heteroaralkyl; or
   W and R₆ taken together with the nitrogen atom to which they are attached form a 8- to 12-membered bicyclic ring, which may be optionally substituted or may contain another heteroatom selected from oxygen, nitrogen and sulfur;
X₂ is —C(R₉)₂—, O, S or —NR₁₀— in which
   R₉ is hydrogen or lower alkyl;
   R₁₀ is hydrogen, alkyl or aralkyl;
      provided that W is not 2-methylquinolin-4-yl when Z is O, p is 1, Q is a bond, X₂ is —C(R₉)₂— in which R₉ is hydrogen, and X₁ is located at the 4-position; or W is not 2-butyl-4-chloro-5-hydroxymethylimidazol-1-yl when Z is a bond, p is 1, Q is a bond, X₂ is —NR₁₀— in which R₁₀ is hydrogen, and X₁ is located at the 4-position;
      or an optical isomer thereof; or a mixture of optical isomers thereof; or a pharmaceutically acceptable salt thereof.

Preferred are the compounds of formula (I) having the formula 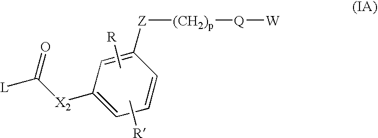
wherein L is a radical selected from the group consisting of: 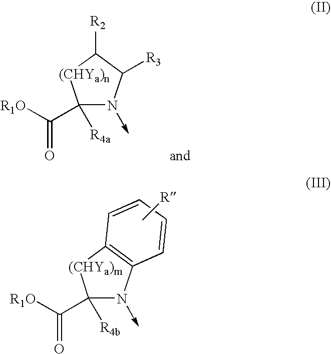
in which
R₁ is hydrogen, optionally substituted alkyl, aryl, heteroaryl, aralkyl or cycloalkyl;
R₂ is hydrogen, hydroxy, oxo, optionally substituted alkyl, aryl, aralkyl, alkoxy, aryloxy, aralkoxy, alkylthio, arylthio or aralkylthio;
R₃ is hydrogen; or
R₂ and R₃ combined are alkylene which together with the carbon atoms to which they are attached form a fused 5- to 7-membered ring; or
R₂ and R₃ combined are a bond between the carbon atoms to which they are attached;
n is 1;
Yₐ is hydrogen; or
Yₐ and R₂ combined are a bond between the carbon atoms to which they are attached;
R₄ₐ is hydrogen; or
R₄ₐ and Yₐ combined are a bond between the carbon atoms to which they are attached;
R″ is hydrogen, optionally substituted alkyl, alkoxy or halogen;
m is 1;
Y_{b} is hydrogen;
R_{4b} is hydrogen; or
R_{4b} and Y_{b} combined are a bond between the carbon atoms to which they are attached;
R and R′ are independently hydrogen, halogen, optionally substituted alkyl, alkoxy, aralkyl or heteroaralkyl; or
R and R′ combined together with the carbon atoms to which they are attached form an optionally substituted fused 5- to 6-membered aromatic or heteroaromatic ring provided that R and R′ are attached to carbon atoms adjacent to each other; or
Z is a bond, O or S;
p is an integer from 1 to 8;
Q is a bond; or
Q is —O(CH₂)ᵣ— or —S(CH₂)ᵣ— in which
   r is zero or an integer from 1 to 8; or
Q is —C(O)NR₆— in which
   R₆ is hydrogen, optionally substituted alkyl, cycloalkyl, aryl, heteroaryl, aralkyl or heteroaralkyl; or
Q is —NR₇—, —NR₇C(O)—, —NR₇C(O)NR₈— or —NR₇C(O)O— in which
   R₇ is hydrogen, optionally substituted alkyl, cycloalkyl, aryl, heteroaryl, aralkyl or heteroaralkyl;
   R₈ is hydrogen, alkyl or aralkyl;
W is cycloalkyl, aryl, heterocyclyl, aralkyl or heteroaralkyl; or
W and R₆ taken together with the nitrogen atom to which they are attached form a 8- to 12-membered bicyclic ring, which may be optionally substituted or may contain another heteroatom selected from oxygen, nitrogen and sulfur;
X₂ is —C(R₉)₂—, O, S or —NR₁₀— in which
   R₉ is hydrogen or lower alkyl;
   R₁₀ is hydrogen or lower alkyl;
      or an optical isomer thereof; or a mixture of optical isomers thereof; or a pharmaceutically acceptable salt thereof.

Preferred are the compounds of formula (IA) wherein
R₁ is hydrogen or optionally substituted alkyl;
R₂ and R₃ are hydrogen;
Yₐ and Y_{b} are hydrogen;
R₄ₐ and R_{4b} are hydrogen;
R and R′ are independently hydrogen, halogen, optionally substituted C₁₋₆ alkyl or C₁₋₆ alkoxy;
p is an integer from 1 to 5;
Q is a bond; or
Q is —O(CH₂)ᵣ— or —S(CH₂)ᵣ— in which
   r is zero or 1; or
Q is —C(O)NR₆— in which
   R₆ is hydrogen or lower alkyl; or
Q is —NR₇—, —NR₇C(O)—, —NR₇C(O)NR₈— or —NR₇C(O)O— in which
   R₇ is hydrogen or optionally substituted alkyl;
   R₈ is hydrogen or alkyl;
X₂ is —C(R₉)₂—, O, S or —NR₁₀— in which
   R₉ is hydrogen or methyl;
   R₁₀ is hydrogen;
      or an optical isomer thereof; or a mixture of optical isomers thereof; or a pharmaceutically acceptable salt thereof.

More preferred are the compounds of formula (IA) wherein
R, R′ and R″ are hydrogen;
Q is a bond; or
Q is —O(CH₂)ᵣ— or —S(CH₂)ᵣ— in which
   r is zero; or
Q is —NR₇—, —NR₇C(O)—, —NR₇C(O)NR₈— or —NR₇C(O)O— in which
   R₇ is hydrogen or optionally substituted lower alkyl;
W is cycloalkyl, aryl or heterocyclyl;
   or an optical isomer thereof; or a mixture of optical isomers thereof; or a pharmaceutically acceptable salt thereof.

Most preferred are the compounds of formula (IA), wherein the asymmetric center in radical L is in the (R) configuration; or a pharmaceutically acceptable salt thereof.

Most preferred are also the compounds of formula (IA), wherein X₂ is —C(R₉)₂— in which R₉ is methyl; or an optical isomer thereof; or a mixture of optical isomers thereof; or a pharmaceutically acceptable salt thereof.

Most preferred are also the compounds of formula (IA) having the formula 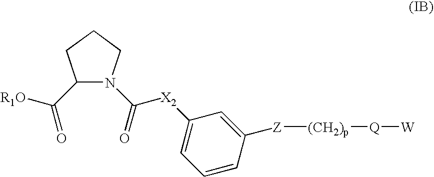
wherein
R₁ is hydrogen or optionally substituted alkyl;
Z is a bond, O or S;
p is an integer from 1 to 3;
Q is a bond, O or S; or
Q is —NR₇C(O)— in which
   R₇ is hydrogen or optionally substituted lower alkyl;
W is aryl or heterocyclyl;
X₂ is —C(R₉)₂—, O, S or —NH— in which
   R₉ is hydrogen or methyl;
      or an optical isomer thereof; or a mixture of optical isomers thereof; or a pharmaceutically acceptable salt thereof.

Preferred are the compounds of formula (IB) wherein
Z is O or S;
p is an integer of 2 or 3;
Q is O or S;
W is selected from the group consisting of: 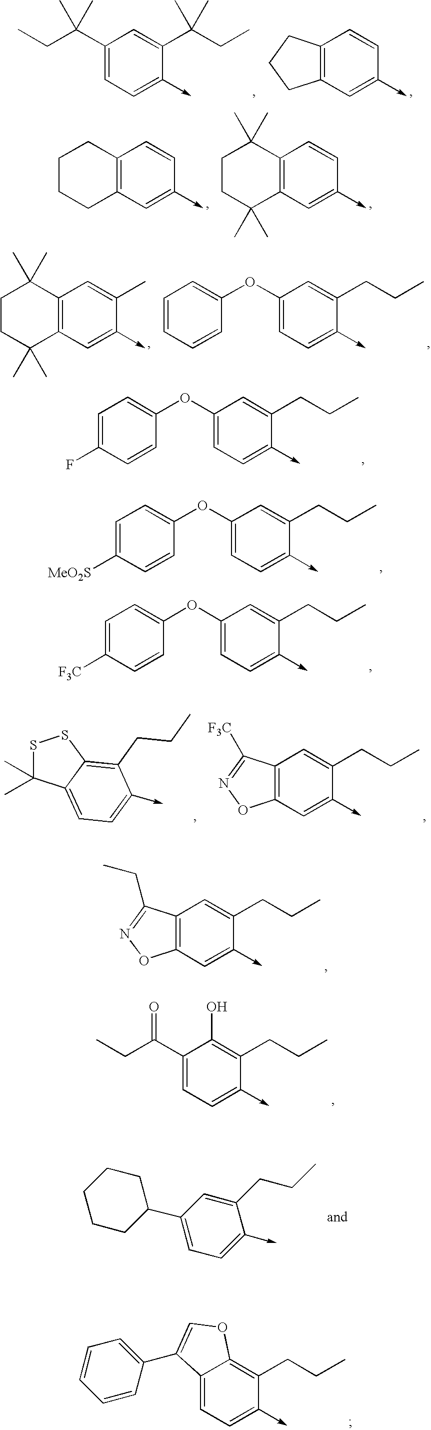
   or an optical isomer thereof; or a mixture of optical isomers thereof; or a pharmaceutically acceptable salt thereof.

Preferred are also the compounds of formula (IB), designated as the A group, wherein
Z is bond, O or S;
p is an integer of 1 or 2;
Q is a bond;
W is selected from the group consisting of: 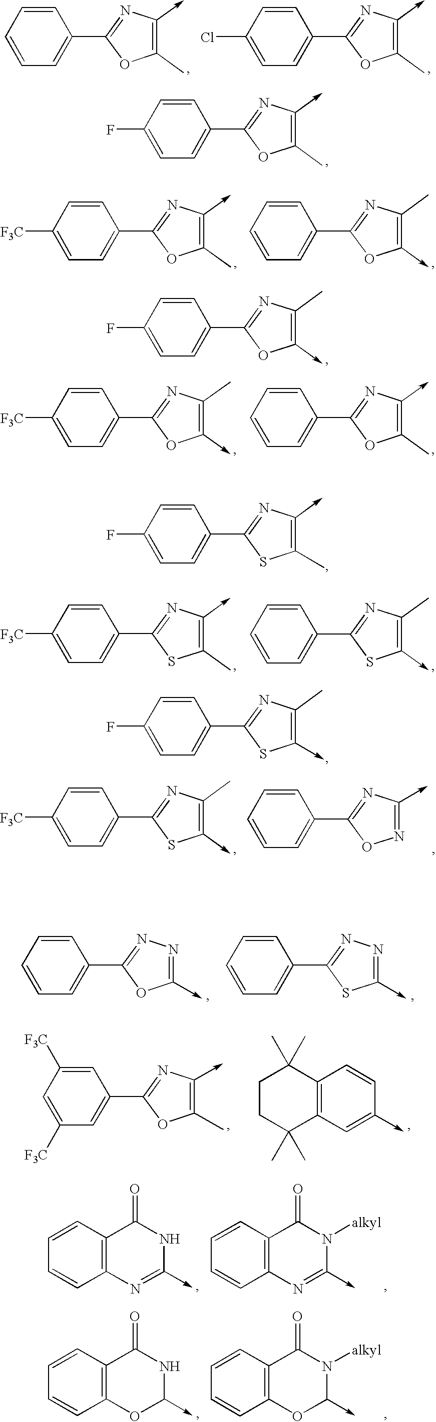 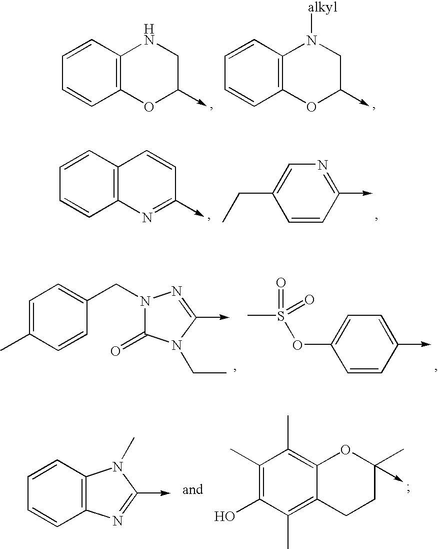
   or an optical isomer thereof; or a mixture of optical isomers thereof; or a pharmaceutically acceptable salt thereof.

Preferred are the compounds in the A group wherein
Z is O;
p is 1;
X₂ is —C(R₉)₂— in which R₉ is methyl;
W is selected from the group consisting of: 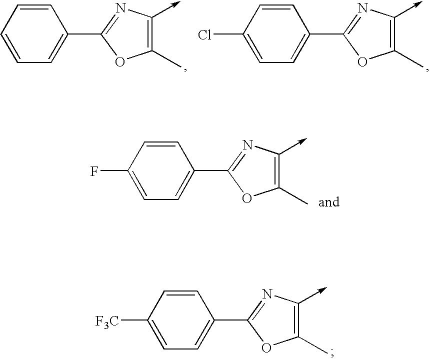
   or an optical isomer thereof; or a mixture of optical isomers thereof; or a pharmaceutically acceptable salt thereof.

Further preferred are the compounds in the A group wherein the asymmetric center in radical L is in the (R) configuration; or a pharmaceutically acceptable salt thereof.

Preferred are also the compounds of formula (IB) wherein
Z is O or S;
p is 2;
Q is a bond;
W is selected from the group consisting of: 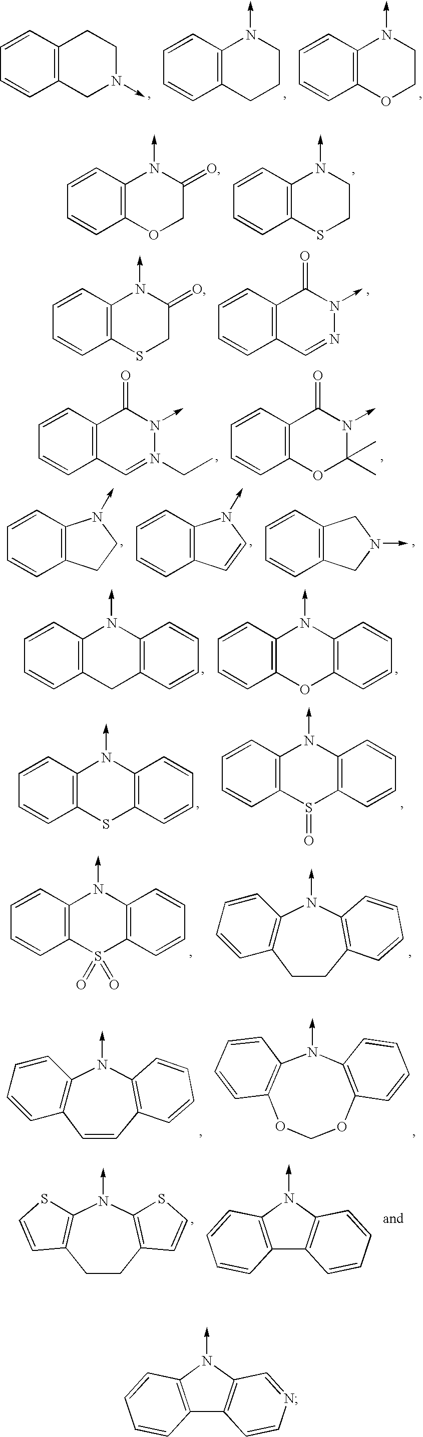
   or an optical isomer thereof; or a mixture of optical isomers thereof; or a pharmaceutically acceptable salt thereof.

Preferred are also the compounds of formula (IB) wherein
Z is a bond;
p is 1;
Q is —NR₇C(O)— in which
   R₇ is hydrogen or methyl;
W is selected from the group consisting of: 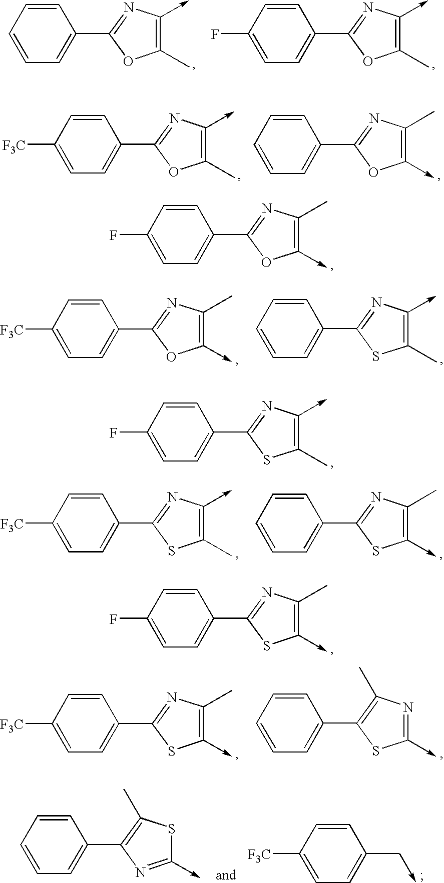
   or an optical isomer thereof; or a mixture of optical isomers thereof; or a pharmaceutically acceptable salt thereof.

Most preferred are also the compounds of formula (IA) having the formula 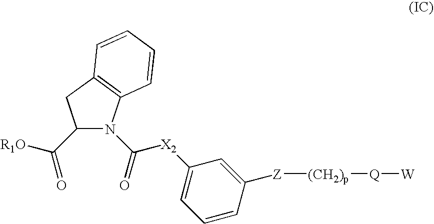
wherein
R₁ is hydrogen or optionally substituted alkyl;
Z is a bond, O or S;
p is an integer from 1 to 3;
Q is a bond, O or S; or
Q is —NR₇C(O)— in which
   R₇ is hydrogen or optionally substituted lower alkyl;
W is aryl or heterocyclyl;
X₂ is —C(R₉)₂—, O, S or —NH— in which
   R₉ is hydrogen or methyl;
      or an optical isomer thereof; or a mixture of optical isomers thereof; or a pharmaceutically acceptable salt thereof.

Preferred are the compounds of formula (IC) wherein
Z is O or S;
p is an integer of 2 or 3;
Q is O or S;
W is selected from the group consisting of: 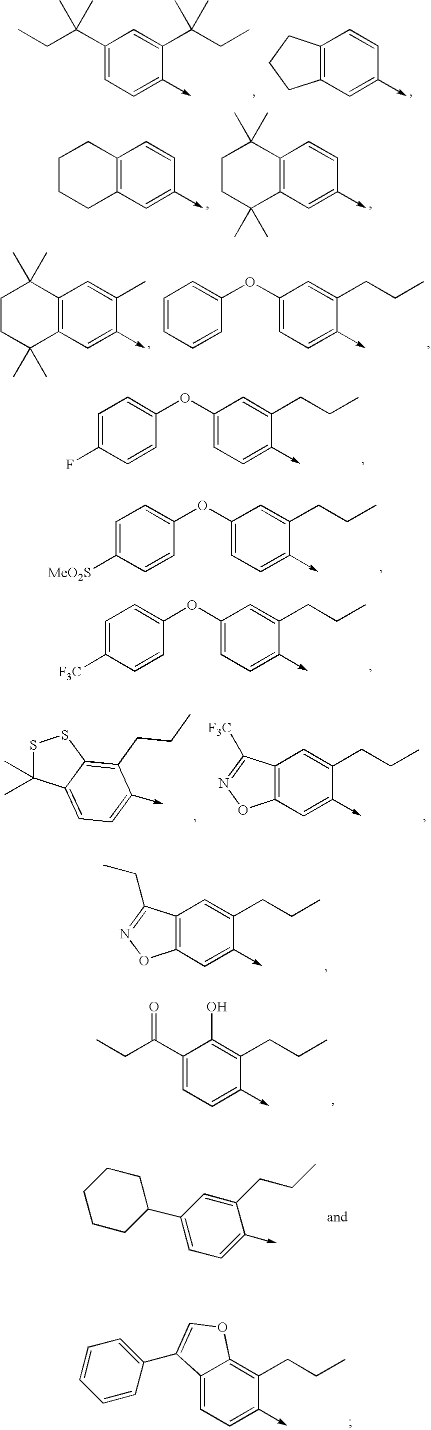
   or an optical isomer thereof; or a mixture of optical isomers thereof; or a pharmaceutically acceptable salt thereof.

Preferred are also the compounds of formula (IC), designated as the B group, wherein
Z is bond, O or S;
p is an integer of 1 or 2;
Q is a bond;
W is selected from the group consisting of: 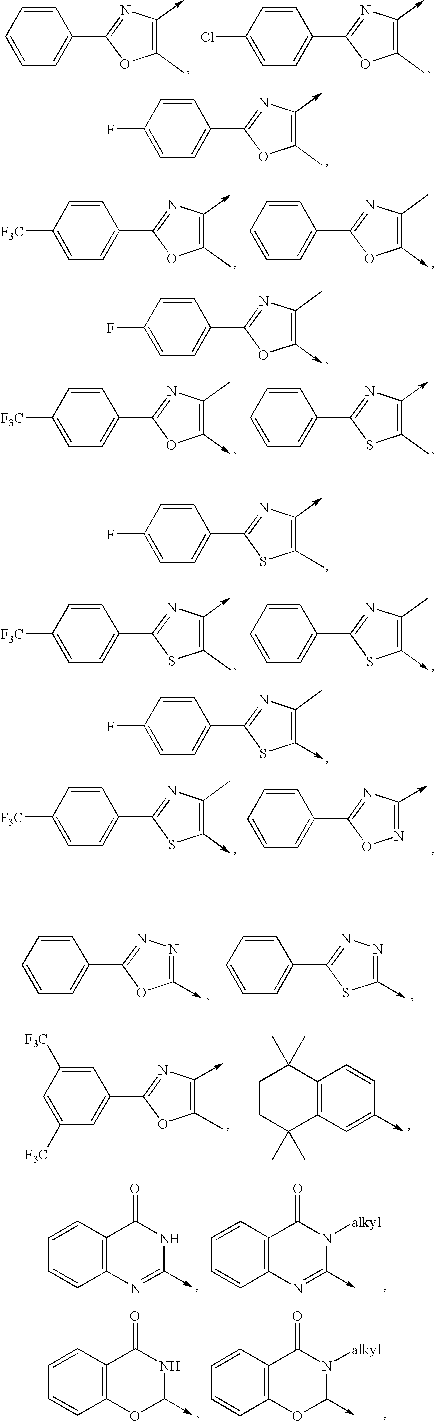 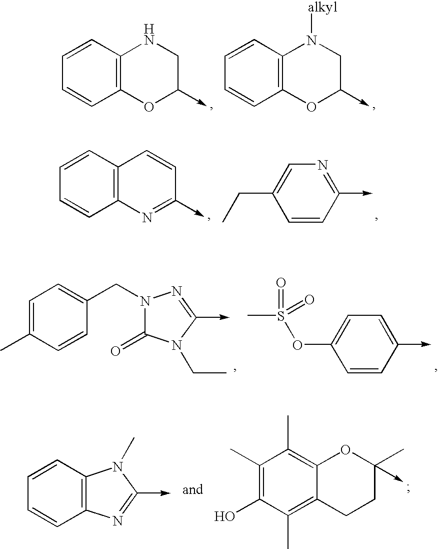
   or an optical isomer thereof; or a mixture of optical isomers thereof; or a pharmaceutically acceptable salt thereof.

Preferred are the compounds in the B group wherein
Z is O;
p is 1;
X₂ is —C(R₉)₂— in which R₉ is methyl;
W is selected from the group consisting of: 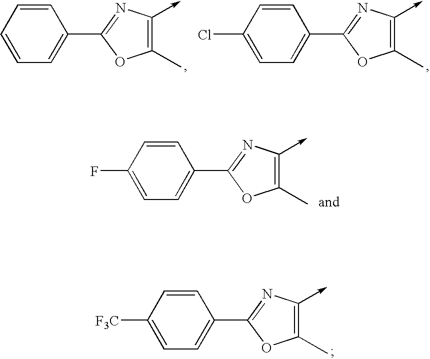
   or an optical isomer thereof; or a mixture of optical isomers thereof; or a pharmaceutically acceptable salt thereof.

Further preferred are the compounds in the B group wherein the asymmetric center in radical L is in the (R) configuration; or a pharmaceutically acceptable salt thereof.
Preferred are also the compounds of formula (IC) wherein
Z is O or S;
p is 2;
Q is a bond;
W is selected from the group consisting of: 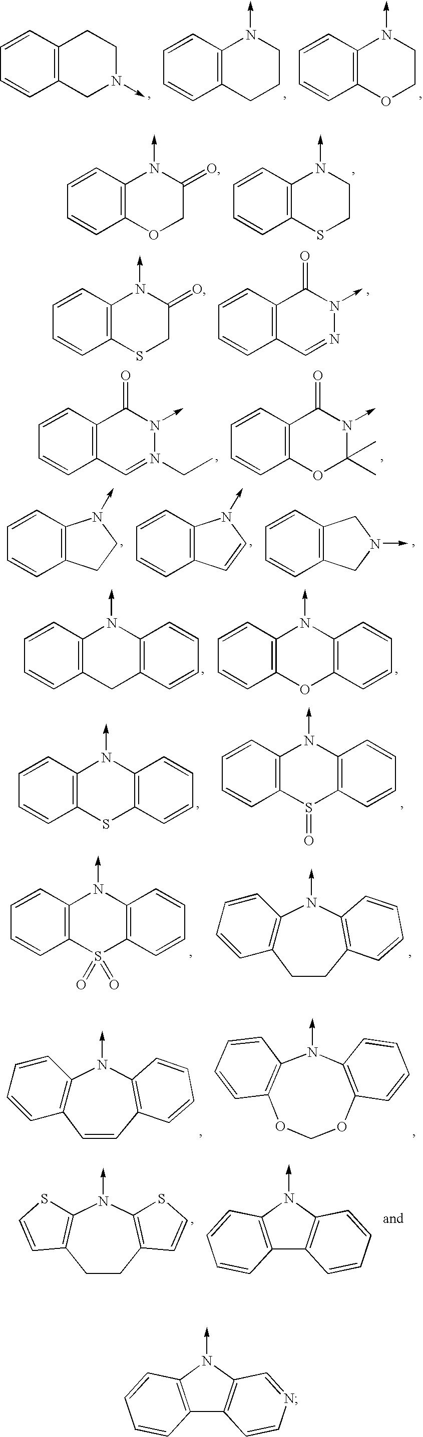
   or an optical isomer thereof; or a mixture of optical isomers thereof; or a pharmaceutically acceptable salt thereof.

Preferred are also the compounds of formula (IC) wherein
Z is a bond;
p is 1;
Q is —NR₇C(O)— in which
   R₇ is hydrogen or methyl;
W is selected from the group consisting of: 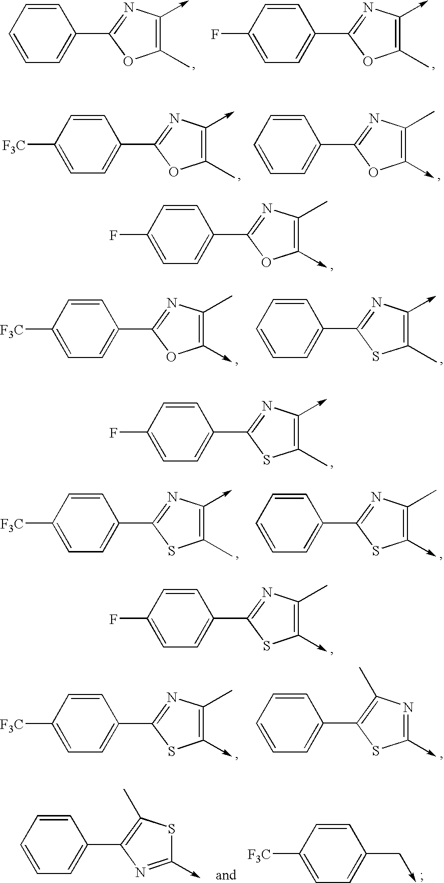
   or an optical isomer thereof; or a mixture of optical isomers thereof; or a pharmaceutically acceptable salt thereof.

Particular embodiments of the invention are:
(R)-1-{2-[3-(5-Methyl-2-phenyl-oxazol-4-ylmethoxy)-phenyl]-acetyl}-pyrrolidine-2-carboxylic acid;
(R)-1-[3-(5-Methyl-2-phenyl-oxazol-4-ylmethoxy)-phenylsulfanylcarbonyl]- pyrrolidine-2-carboxylic acid;
(R)-Pyrrolidine-1,2-dicarboxylic acid-1-[3-(5-methyl-2-phenyl-oxazol-4-ylmethoxy)-phenyl]ester;
(R)-1-{2-Methyl-2-[3-(5-methyl-2-phenyl-oxazol-4-ylmethoxy)-phenyl]-propionyl}-pyrrolidine-2-carboxylic acid;
(R)-1-{2-[4-(5-Methyl-2-phenyl-oxazol-4-ylmethoxy)-phenyl]-acetyl}-pyrrolidine-2-carboxylic acid;
(R)-1-{2-[4-(5-Methyl-2-phenyl-oxazol-4-ylmethoxy)-phenyl]-acetyl}-pyrrolidine-2-carboxylic acid;
(R)-1-(2-{3-[2-(4-Carbamoylphenyl)-5-methyl-oxazol-4-ylmethoxy]-phenyl}-2-methyl-propionyl)-pyrrolidine-2-carboxylic acid;
(R)-1-(2-{3-[2-(4-Cyano-phenyl)-5-methyl-oxazol-4-ylmethoxy]phenyl}-2-methyl-propionyl)-pyrrolidine-2-carboxylic acid;
(R)-1-(2-{3-[2-(4-Chloro-3-fluoro-phenyl)-5-methyl-oxazol-4-yl-methoxy]-phenyl}-2-methyl-propionyl)-pyrrolidine-2-carboxylic acid;
(R)-1-{2-Methyl-2-[4-({methyl-[2-(4-trifluoromethyl-phenyl)-acetyl]-amino}-methyl)-phenyl]-propionyl}-pyrrolidine-2-carboxylic acid;
(R)-1-(2-{3-[2-(4-Fluoro-phenyl)-5-methyl-oxazol-4-ylmethoxy]-4-methoxy-phenyl}-2-methyl-propionyl)-pyrrolidine-2-carboxylic acid;
(R)-1-(2-{3-[2-(4-Chloro-phenyl)-5-methyl-oxazol-4-ylmethoxy]-phenyl}-2-methyl-propionyl)-pyrrolidine-2-carboxylic acid;
(R)-1-{2-Methyl-2-[3-(5-methyl-2-p-tolyl-oxazol-4-ylmethoxy)-phenyl]-propionyl}-pyrrolidine-2-carboxylic acid;
(R)-1-[2-(4-{2-[2-(4-Trifluoromethyl-phenyl)-acetylamino]-ethyl}-phenyl)-acetyl]-pyrrolidine-2-carboxylic acid;
(R)-1-(2-Methyl-2-{3-[5-methyl-2-(4-trifluoromethyl-phenyl)-oxazol-4-ylmethoxy]-phenyl}-propionyl)-pyrrolidine-2-carboxylic acid;
(R)-1-(2-{3-[2-(4-Fluoro-phenyl)-5-methyl-oxazol-4-ylmethoxy]-phenyl}-2-methyl-propionyl)-pyrrolidine-2-carboxylic acid;
(R)-1-(2-{3-[2-(5-Methyl-2-phenyl-oxazol-4-yl)-ethyl]-phenyl}-acetyl)-pyrrolidine-2-carboxylic acid;
(R)-1-[2-(3-{[(4-Methyl-5-phenyl-thiazole-2-carbonyl)-amino]-methyl}-phenyl)-acetyl]-pyrrolidine-2-carboxylic acid;
(R)-1-[2-Methyl-2-(3-{[(4-methyl-2-phenyl-thiazole-5-carbonyl)-amino]-methyl}-phenyl)-propionyl]-pyrrolidine-2-carboxylic acid;
(R)-1-[2-(3-{[(4-Methyl-2-phenyl-thiazole-5-carbonyl)-amino]-methyl}-phenyl)-acetyl]-pyrrolidine-2-carboxylic acid;
(R)-1-{2-[3-(1-Benzyl-4-ethyl-5-oxo-4,5-dihydro-1H-[1,2,4]triazol-3-ylmethoxy)-phenyl]-acetyl}-pyrrolidine-2-carboxylic acid;
(R)-1-(2-{3-[2-(5-Methyl-2-phenyl-oxazol-4-yl)-ethoxy]-phenyl}-acetyl)-pyrrolidine-2-carboxylic acid;
(R)-1-(2-{3-[5-Methyl-2-(4-trifluoromethyl-phenyl)-oxazol-4-ylmethoxy]-phenyl}-acetyl)-pyrrolidine-2-carboxylic acid;
(S)-1-{2-[3-(5-Methyl-2-phenyl-oxazol-4-ylmethoxy)-phenyl]-acetyl}-pyrrolidine-2-carboxylic acid;
(R)-1-{2-[3-(4-Methyl-benzyloxy)-phenyl]-acetyl}-pyrrolidine-2-carboxylic acid;
(R)-1-{2-Methyl-2-[3-(5-methyl-2-phenyl-oxazol-4-ylmethoxy)-phenyl]-propionyl}-2,3-dihydro-1H-indole-2-carboxylic acid;
(R)-1-(2-{3-[2-(4-Carbamoyl-phenyl)-5-methyl-oxazol-4-ylmethoxy]-phenyl}-2-methyl-propionyl)-2,3-dihydro-1H-indole-2-carboxylic acid;
(R)-1-(2-{3-[2-(4-Chloro-3-fluoro-phenyl)-5-methyl-oxazol-4-ylmethoxy]-phenyl}-2-methyl-propionyl)-2,3-dihydro-1H-indole-2-carboxylic acid;
(R)-1-(2-{3-[2-(4-Cyano-phenyl)-5-methyl-oxazol-4-ylmethoxy]-phenyl}-2-methyl-propionyl)-2,3-dihydro-1H-indole-2-carboxylic acid;
(R)-1-(2-{3-[2-(4-Fluoro-phenyl)-5-methyl-oxazol-4-ylmethoxy]-4-methoxy-phenyl}-2-methyl-propionyl)-2,3-dihydro-1H-indole-2-carboxylic acid;
(R)-1-{2-Methyl-2-[3-(5-methyl-2-p-tolyl-oxazol-4-ylmethoxy)-phenyl]-propionyl}-2,3-dihydro-1H-indole-2-carboxylic acid;
(R)-1-(2-Methyl-2-{3-[5-methyl-2-(4-trifluoromethyl-phenyl)-oxazol-4-ylmethoxy]-phenyl}-propionyl)-2,3-dihydro-1H-indole-2-carboxylic acid;
(R)-1-(2-{3-[2-(4-Chloro-phenyl)-5-methyl-oxazol-4-ylmethoxy]-phenyl}-2-methyl-propionyl)-2,3-dihydro-1H-indole-2-carboxylic acid; and
(R)-1-(2-{3-[2-(4-Fluoro-phenyl)-5-methyl-oxazol-4-ylmethoxy]-phenyl}-2-methyl-propionyl)-2,3-dihydro-1H-indole-2-carboxylic acid;
   or an optical isomer thereof; or a mixture of optical isomers thereof; or a pharmaceutically acceptable salt thereof.

Methods of preparing the above compounds are disclosed in WO 04/103995 published Dec. 2, 2004, which is incorporated herein in its entirety.

Dual acting PPAR alpha/gamma agonists include those disclosed in co-owned international application PCT/EP02/13025 published on May 30, 2003 with publication No. WO 03/043985, particularly compound 19 of Example 4, shown as compound 4-19, formula

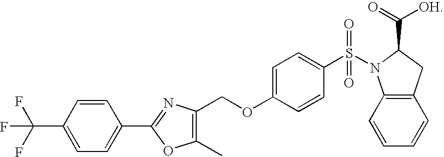

HDL increasing compounds include but are not limited to cholesterol ester transfer protein inhibitors (CETP inhibitor). Examples of CETP inhibitors include JTT705 disclosed in example 26 of U.S. Pat. No. 6,426,365 issued Jul. 30, 2002 and pharmaceutically acceptable salts thereof.

Anti-diabetics include PPAR delta compounds; insulin sensitivity enhancers which restore impaired insulin receptor function to reduce insulin resistance and consequently enhance the insulin sensitivity.

Examples of PPAR delta agonists include the compounds of formula

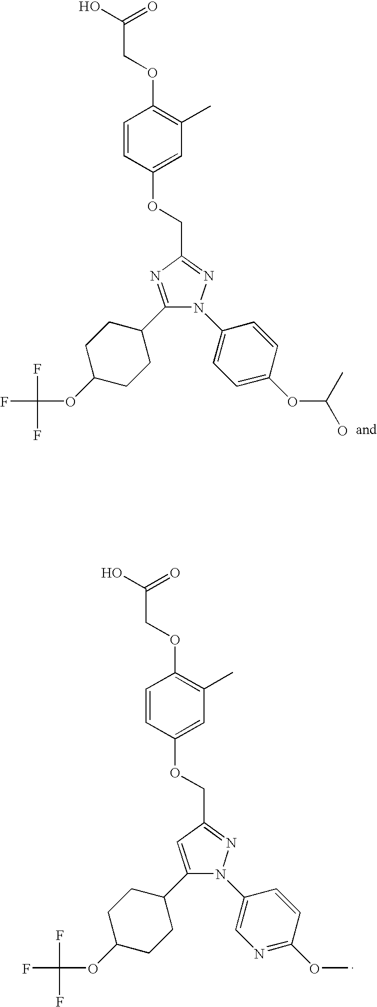

An appropriate insulin sensitivity enhancer is, for example, an appropriate hypoglycemic thiazolidinedione derivative (glitazone).

An appropriate glitazone is, for example, (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297). Preferred are pioglitazone, rosiglitazone and troglitazone.

Anti-diabetics include non-glitazone type PPARγ agonists, especially N-(2-benzoylphenyl)-L-tyrosine analogues, e.g. GI-262570, and JTT501.

Anti-hypertensive agents include angiotensin converting enzyme inhibitors (ACE-inhibitors);
renin inhibitors, calcium channel blockers, diuretics, beta-blockers, neutral endo-peptidase inhibitors (NEP inhibitors), endothelin converting enzyme inhibitors (ECE inhibitors) and AT₁ receptor antagonists, optionally in combination with a diuretic, for example, Co-Diovan®. The interruption of the enzymatic degradation of angiotensin I to angiotensin II with ACE-inhibitors is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril (cf. EP 7477), benazepril (cf. EP 72352), benazeprilat (cf. EP 72352), captopril (cf. U.S. Pat. No. 4,105,776), ceronapril (cf. EP 229520), cilazapril (cf. EP 94095), delapril (cf. EP 51391), enalapril (cf. EP 12401), enaprilat (cf. EP 12401), fosinopril (cf. EP 53902), imidapril (cf. EP 95163), lisinopril (cf. EP 12401), moveltipril (cf. ZA 82/3779), perindopril (cf. EP 49658), quinapril (cf. EP 49605), ramipril (cf. EP 79022), spirapril (cf. EP 50800), temocapril (cf. EP 161801), and trandolapril (cf. EP 551927), or, in each case, a pharmaceutically acceptable salt thereof.

Preferred AGE inhibitors are those agents that have been marketed, most preferred are benazepril and enalapril.

Comprised are likewise the corresponding stereoisomers as well as the corresponding crystal modifications, e.g. solvates and polymorphs, which are disclosed therein and, where applicable, all pharmaceutically acceptable salts thereof.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

The class of AT₁ receptor antagonists comprises compounds having differing structural features, essentially preferred are the non-peptidic ones. For example, mention may be made of the compounds which are selected from the group consisting of valsartan (cf. EP 443983), losartan (cf. EP253310), candesartan (cf. 459136), eprosartan (cf. EP 403159), irbesartan (cf. EP454511), olmesartan (cf. EP 503785), tasosartan (cf. EP539086), telmisartan (cf. EP 522314), the compound with the designation E-4177 of the formula 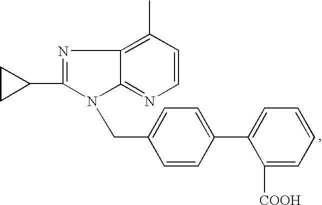
the compound with the designation SC-52458 of the following formula 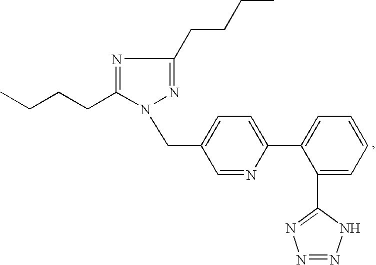
and the compound with the designation the compound ZD-8731 of the following formula 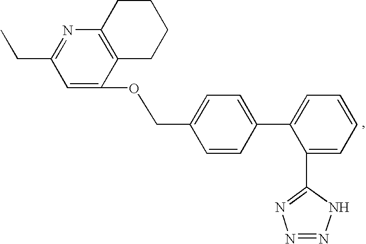
or, in each case, a pharmaceutically acceptable salt thereof.

Preferred AT₁-receptor antagonist are those agents which have been marketed, most preferred is Diovan® and Co-Diovan® or a pharmaceutically acceptable salt thereof.

The class of CCBs essentially comprises dihydropyridines (DHPs) and non-DHPs, such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g., as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine and verapamil, or, e.g., dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

A diuretic is, e.g., a thiazide derivative selected from the group consisting of chlorothiazide, hydrochlorothiazide, methylclothiazide, amiloride, triamterene and chlorothalidon. The most preferred is hydrochlorothiazide.

Beta-blockers suitable for use in the present invention include beta-adrenergic blocking agents (beta-blockers) which compete with epinephrine for beta-adrenergic receptors and interfere with the action of epinephrine. Preferably, the beta-blockers are selective for the beta-adrenergic receptor as compared to the alpha-adrenergic receptors, and so do not have a significant alpha-blocking effect. Suitable beta-blockers include compounds selected from acebutolol, atenolol, betaxolol, bisoprolol, carteolol, carvedilol, esmolol, labetalol, metoprolol, nadolol, oxprenolol, penbutolol, pindolol, propranolol, sotalol and timolol. Where the beta-blocker is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug, such as a physiologically hydrolizable and acceptable ester. For example, metoprolol is suitably administered as its tartrate salt, propranolol is suitably administered as the hydrochloride salt, and so forth.

NEP inhibitors within the scope of the present invention include compounds disclosed in U.S. Pat. Nos. 5,223,516 and 4,610,816, herein incorporated by reference, including in particular N—[N-[1(S)-carboxyl-3-phenylproplyl]-(S)-phenylalanyl]-(S)-isoserine and N—[N-[((1S)-carboxy-2-phenyl)ethyl]-(S)-phenylalanyl]-β-alanine; compounds disclosed in U.S. Pat. No. 4,929,641, in particular N-[2(S)-mercaptomethyl-3-(2-methylphenyl)-propionyl]methionine; SQ 28603 (N-[2-(mercaptomethyl)-1-oxo-3-phenylpropyl]-β-alanine), disclosed in South African Patent Application 84/0670; UK 69578 (cis-4-[[[1-[2-carboxy-3-(2-methoxyethoxy)propyl]-cyclopentyl]carbonyl]amino]-cyclohexanecarboxylic acid) and its active enantiomer(s); thiorphan and its enantiomers; retro-thiorphan; phosphoramidon; and SQ 29072 (7-[[2-(mercaptomethyl)-1-oxo-3-phenylpropyl]amino]-heptanoic acid). Also suitable for use are any pro-drug forms of the above-listed NEP inhibitors, e.g., compounds in which one or more carboxylic acid groups are esterified.

NEP inhibitors within the scope of the present invention also include the compounds disclosed in U.S. Pat. No. 5,217,996, particularly, N-(3-carboxy-1-oxopropyl)-(4S)-p-phenylphenylmethyl)-4-amino-2R-methylbutanoic acid ethyl ester; the compounds disclosed in EP 00342850, particularly (S)-cis-4-[1-[2-(5-indanyloxycarbonyl)-3-(2-methoxyethoxy)propyl]-1-cyclopentanecarboxamido]-1-cyclohexanecarboxylic acid; the compounds disclosed in GB 02218983, particularly 3-(1-[6-endo-hydroxymethylbicyclo[2,2,1]heptane-2-exo-carbamoyl]cyclopentyl)-2-(2-methoxyethyl)propanoic acid; the compounds disclosed in WO 92/14706, particularly N-(1-(3-(N-t-butoxycarbonyl-(S)-propylamino)-2(S)-t-butoxy-carbonylpropyl)cyclopentanecarbonyl)-O-benzyl-(S)-serine methyl ester; the compounds disclosed in EP 00343911; the compounds disclosed in JP 06234754; the compounds disclosed in EP 00361365, particularly 4-[[2-(Mercaptomethyl)-1-oxo-3-phenylpropyl]amino]benzoic acid; the compounds disclosed in WO 90/09374, particularly 3-[1-(Cis-4-carboxycarbonyl-cis-3-butylcyclohexyl-r-1-carbamoyl)cyclopentyl]-2S-(2-methoxyethoxymethyl)propanoic acid; the compounds disclosed in JP 07157459, particularly N-((2S)-2-(4-biphenylmethyl)-4-carboxy-5-phenoxyvaleryl)glycine; the compounds disclosed in WO 94/15908 particularly N-(1-(N-hydroxycarbamoylmethyl)-1-cyclopentanecarbonyl)-L-phenylalanine; the compounds disclosed in U.S. Pat. No. 5,273,990 particularly (S)-(2-biphenyl-4-yl)-1-(1H-tetrazol-5-yl)ethylamino)methylphosphonic acid; the compounds disclosed in U.S. Pat. No. 5,294,632 particularly (S)-5-(N-(2-(phosphonomethylamino)-3-(4-biphenyl)propionyl)-2-aminoethyl)tetrazole; the compounds disclosed in U.S. Pat. No. 5,250,522, particularly β-Alanine, 3-[1,1′-biphenyl]-4-yl-N-[diphenoxyphosphinyl)methyl]-L-alanyl; the compounds disclosed in EP 00636621, particularly N-(2-carboxy-4-thienyl)-3-mercapto-2-benzylpropanamide; the compounds disclosed in WO 93/09101, particularly 2-(2-mercaptomethyl-3-phenylpropionamido)thiazol-4-ylcarboxylic acid; the compounds disclosed in EP 00590442 particularly ((L)-(1-((2,2-dimethyl-1,3-dioxolan-4-yl)-methoxy)carbonyl)-2-phenylethyl)-L-phenylalanyl)-β-alanine, N—[N-[(L)-[1-[(2,2-dimethyl-1,3-dioxolan-4-yl)-methoxy]carbonyl]-2-phenylethyl]-L-phenylalanyl]-(R)-alanine, N-1-[N-[(L)-1-carboxy-2-phenylethyl]-L-phenylalanyl]-(R)-alanine, N-[2-acetylthiomethyl-3-(2-methyl-phenyl)propionyl]-methionine ethyl ester, N-[2-mercaptomethyl-3-(2-methylphenyl)propioyl]-methionine, N-[2(S)-mercaptomethyl-3-(2-methylphenyl)propanoyl]-(S)-isoserine, N—(S)-[3-mercapto-2-(2-methylphenyl)propionyl]-(S)-2-methoxy-(R)-alanine, N-[1-[[1(S)-benzyoxycarbonyl-3-phenylpropyl]amino]cyclopentylcarbonyl]-(S)-isoserine, N-[1-[[1(S)-carbonyl-3-phenylpropy]amino]-cyclopentylcarbonyl]-(S)-isoserine, 1,1′-[dithiobis-[2(S)-(2-methylbenzyl)-1-oxo-3,1-propanediyl]]-bis-(S)-isoserine, 1,1′-[dithiobis-[2(S)-(2-methylbenzyl)-1-oxo-3,1-propanediyl]]-bis-(S)-methionine, N-(3-phenyl-2-(mercaptomethyl)-propionyl)-(S)-4-(methylmercapto)methionine, N-[2-acetylthiomethyl-3-phenyl-propionyl]-3-aminobenzoic acid, N-[2-mercaptomethyl-3-phenyl-propionyl]-3-aminobenzoic acid, N-[1-(2-carboxy-4-phenylbutyl)-cyclopentanecarbonyl]-(S)-isoserine, N-[1-(acetylthiomethyl)cyclopentane-carbonyl]-(S)-methionine ethyl ester, 3(S)-[2-(acetylthiomethyl)-3-phenyl-propionyl]amino-ε-caprolactam; and the compounds disclosed in WO 93/10773 particularly N-(2-acetylthiomethyl-3-(2-methylphenyl)propionyl)-methionine ethyl ester.

ECE inhibitors include SLV306.

Renin inhibitors comprise, e.g., peptidic and, preferably, non-peptidic renin inhibitors.

A non-peptidic renin inhibitor is, e.g., ditekiren, terlakiren, zankiren, SPP-100 or a compound of formula (I) 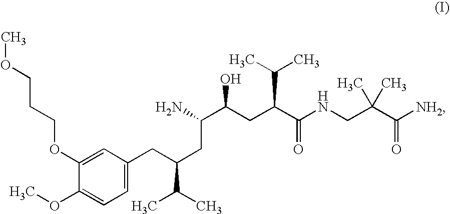
or, in each case, a pharmaceutically acceptable salt thereof.

The renin inhibitor of formula (I), chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, is specifically disclosed in EP-678503 A. Especially preferred is the hemi-fumarate salt thereof.

Non-peptidic renin inhibitor comprise those that are disclosed in WO 97/09311, especially corresponding renin inhibitors as disclosed in the claims and working examples, especially SPP100 of the formula 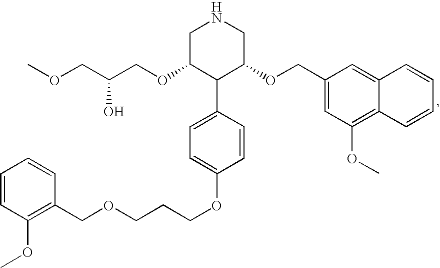
especially and of RO 66-1132 and RO-66-1168 of formula 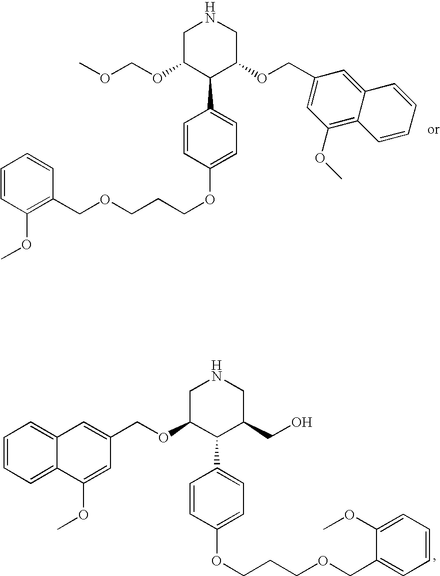
respectively, WO 04/002957, especially those renin inhibitors as disclosed in the working examples and claims. The corresponding subject matter of said WO applications is herein incorporated by reference into the present invention.

Cholesterol absorption modulators include Zetia® and KT6-971 (Kotobuki Pharmaceutical Co. Japan).

Apo-A1 analogs and mimetics include the 18 amino acid D4F peptide as disclosed in Sequence ID No. 5 of U.S. Pat. No. 6,664,230 issued Dec. 16, 2003.

Thrombin inhibitors include Astra Zeneca's Ximelagatran (Exanta®) disclosed in WO 97/23499 published Oct. 12, 1999.

Aldosterone inhibitors include compounds having differing structural features. For example, mention may be made of the compounds which are selected from the group consisting of the non-steroidal aromatase inhibitors anastrozole, fadrozole (including the (+)-enantiomer thereof, as well as the steroidal aromatase inhibitor exemestane, or, in each case where applicable, a pharmaceutically acceptable salt thereof. Also included is epleronone.

The most preferred non-steroidal aldosterone synthase inhibitor is the (+)-enantiomer of the hydrochloride of fadrozole (U.S. Pat. Nos. 4,617,307 and 4,889,861) of formula

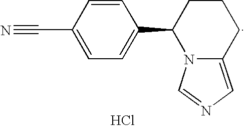

GLP-1 agonists includes GLP-1 analogs, GLP-1 receptor agonists and G-protein coupled receptor 120 (GPR120) agonists. GLP-1 analogs by way of example include Exendin-4™ (exenatide) or LY315902, Myers S R et al., Annual Meeting and Scientific Sessions of the American Diabetes Association, 1998, 58^{th}: Chicago (Abs 0748), and LY307161 Trautman, M., et al, Diabetologia, 2000, 43: Suppl1 (A146). GPR120 agonists include free fatty acids as set forth in Hirasawa, A. et al, Nature Medicine, Vol. 11, No. 1, January 2005.

Glucagon receptor antagonism includes administration of anti-sense molecules, for example RNA and oligonucleotides, to the gene encoding for the glucagon receptor and glucagon receptor antagonists such as, for example, small molecule antagonists which bind to the glucagon receptor and prevent or hinder the binding of natural ligands thereto. Anti-sense technology per se is known in the art. Disclosure of specific anti-sense oligonucleotides (ASOs) and methods used to identify ASOs are disclosed in Sloop, K., et al., The Journal of Clinical Investigation, Vol. 113, No. 11, June 2004, the disclosure of which is hereby incorporated by reference in its entirety as if set forth in full herein.

Cannabinoid receptor 1 (cb1) antagonists include, but are not limited to, compounds selected from Formula Ia, Ib, Ic, Id, Ie, If, Ig and Ih: 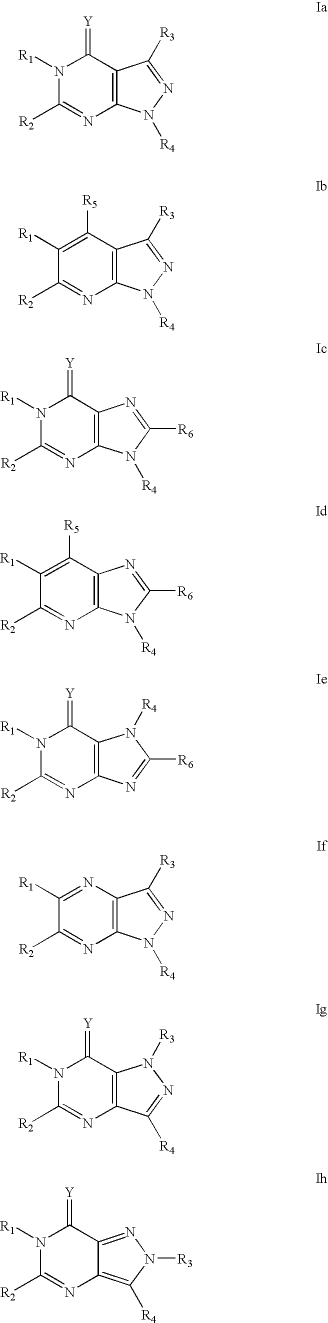
in which:
Y is selected from O, NR₇ and S; wherein R₇ is selected from hydrogen, hydroxy and C₁₋₆alkyl;
R₁ is selected from C₅₋₁₀heteroaryl, C₃₋₁₂cyclolalkyl, phenyl and benzyl; wherein said heteroaryl, cycloalkyl, phenyl and benzyl of R₁ is optionally substituted with 1 to 3 radicals independently selected from halo, hydroxy, cyano, nitro, C₁₋₆alkyl, C₁₋₆alkoxy, halo-substituted C₁₋₆alkyl, halo-substituted C₁₋₆alkoxy, —NR₈R₉, —C(O)OR₈ and R₁₀;
R₂ is selected from C₃₋₈heterocycloalkyl, C₅₋₁₀heteroaryl, phenyl and phenoxy;
wherein said heterocycloalkyl, heteroaryl, phenyl or phenoxy of R₂ is optionally substituted with 1 to 3 radicals independently selected from halo, hydroxy, cyano, nitro, C₁₋₆alkyl, C₁₋₆alkoxy, halo-substituted C₁₋₆alkyl, halo-substituted C₁₋₆alkoxy, —NR₈R₉, —XOR₈, —C(O)R₈, —S(O)₀₋₂R₈, —C(O)NR₈R₉, —C(O)OR₈, —OR₁₀, —NR₈R₁₀ and R₁₀; wherein X is C₁₋₄alkylene;
R₃ is selected from hydrogen, halo, hydroxy, cyano, nitro, C₁₋₆alkyl, C₁₋₆alkoxy, halo-substituted C₁₋₆alkyl, halo-substituted C₁₋₆alkoxy, —NR₈R₉, —C(O)NR₈R₉ and —C(O)OR₈;
R₄ is selected from C₁₋₆alkyl, halo-substituted C₁₋₆alkyl, C₆₋₁₀aryl-C₀₋₄alkyl, C₅₋₁₀heteroaryl, C₃₋₁₂cycloalkyl, C₃₋₈heterocycloalkyl and C(O)R₁₁; wherein R₁₁ is selected from C₃₋₈heterocycloalkyl and C₃₋₈heteroaryl; wherein any alkyl of R₄ can optionally have a methylene replaced with O, S(O)₀₋₂ and NR₈; wherein any cycloalkyl, heterocycloalkyl, aryl or heteroaryl of R₄ can optionally be substituted with 1 to 3 radicals independently selected from halo, hydroxy, cyano, nitro, C₁₋₆alkyl, C₁₋₆alkoxy, halo-substituted-C₁₋₆alkyl, halo-substituted-C₁₋₆alkoxy, XOR₈, S(O)₀₋₂R₈, —NR₈R₉, —C(O)NR₈R₁₀ and —C(O)OR₈;
R₅ is selected from hydrogen, halo, hydroxy, C₁₋₆alkyl, C₁₋₆alkoxy, halo-substituted C₁₋₆alkyl, halo-substituted C₁₋₆alkoxy, —NR₈R₉, —OXOR₈, —OXNR₈R₉ and —C(O)OR₈; wherein X is C₁₋₄alkylene;
R₆ is selected from hydrogen, halo, hydroxy, cyano, nitro, C₁₋₆alkyl, C₁₋₆alkoxy, halo-substituted C₁₋₆alkyl, halo-substituted C₁₋₆alkoxy, —NR₈R₉ and —C(O)OR₈; wherein: R₈ and R₉ are independently selected from hydrogen and C₁₋₆alkyl; or R₈ and R₉ together with the nitrogen atom to which both are attached form C₃₋₈heterocycloalkyl or C₅₋₁₀heteroaryl; and R₁₀ is selected from C₅₋₁₀heteroaryl, C₃₋₈heterocycloalkyl, C₃₋₁₂cycloalkyl and phenyl; wherein said heteroaryl or heterocycloalkyl of R₁₀ or the combination of R₈ and R₉ and additionally the cycloalkyl or phenyl of R₁₀ is optionally substituted with 1 to 3 radicals independently selected from halo, hydroxy, cyano, nitro, C₁₋₆alkyl, C₁₋₆alkoxy, halo-substituted-C₁₋₆alkyl, halo-substituted-C₁₋₆alkoxy, phenyl, —NR₈R₉ and —C(O)OR₈; and the N-oxide derivatives, prodrug derivatives, protected derivatives, individual isomers and mixture of isomers thereof; and the pharmaceutically acceptable salts and solvates (e.g. hydrates) of such compounds; with the proviso that compounds of Formula Ia do not include compounds of Formula II.

Compounds of Formula II are defined as: 5-(4-Isopropyl-phenyl)-1-phenyl-6-p-tolyl-1,5-dihydro-pyrazolo[3,4-d]pyrimidin-4-one; 6-(4-Bromo-phenyl)-1-phenyl-5-p-tolyl-1,5-dihydro-pyrazolo[3,4-d]pyrimidin-4-one; 1-Phenyl-5,6-di-m-tolyl-1,5-dihydro-pyrazolo[3,4-d]pyrimidin-4-one; 1-Phenyl-5,6-di-m-tolyl-1,5-dihydro-pyrazolo[3,4-d]pyrimidin-4-one; 1,5-Diphenyl-6-m-tolyl-1,5-dihydro-pyrazolo[3,4-d]pyrimidin-4-one; 1-Phenyl-5-o-tolyl-6-p-tolyl-1,5-dihydro-pyrazolo[3,4-d]pyrimidin-4-one; 5-(4-Ethoxy-phenyl)-1-phenyl-6-p-tolyl-1,5-dihydro-pyrazolo[3,4-d]pyrimidin-4-one; 5-(4-Chloro-phenyl)-1-phenyl-6-p-tolyl-1,5-dihydro-pyrazolo[3,4-d]pyrimidin-4-one; 5-(4-Isopropyl-phenyl)-1,6-diphenyl-1,5-dihydro-pyrazolo[3,4-d]pyrimidin-4-one; 5-(4-Bromo-phenyl)-6-(2-fluoro-phenyl)-1-phenyl-1,5-dihydro-pyrazolo[3,4-d]pyrimidin-4-one; 5-(4-Methoxy-phenyl)-1-phenyl-6-p-tolyl-1,5-dihydro-pyrazolo[3,4-d]pyrimidin-4-one; 6-(2-Fluoro-phenyl)-1-phenyl-5-m-tolyl-1,5-dihydro-pyrazolo[3,4-d]pyrimidin-4-one; 5-(4-Fluoro-phenyl)-6-(2-fluoro-phenyl)-1-phenyl-1,5-dihydro-pyrazolo[3,4-d]pyrimidin-4-one; 5-(4-Bromo-phenyl)-1-phenyl-6-m-tolyl-1,5-dihydro-pyrazolo[3,4-d]pyrimidin-4-one; 5-(4-Chloro-phenyl)-1-phenyl-6-m-tolyl-1,5-dihydro-pyrazolo[3,4-d]pyrimidin-4-one; 6-(2-Fluoro-phenyl)-5-(4-methoxy-phenyl)-1-phenyl-1,5-dihydro-pyrazolo[3,4-d]pyrimidin-4-one; 1-Phenyl-6-m-tolyl-5-p-tolyl-1,5-dihydro-pyrazolo[3,4-d]pyrimidin-4-one; 6-(4-Chloro-phenyl)-5-(4-fluoro-phenyl)-1-phenyl-1,5-dihydro-pyrazolo[3,4-d]pyrimidin-4-one; 5-(3-Chloro-phenyl)-1-phenyl-6-m-tolyl-1,5-dihydro-pyrazolo[3,4-d]pyrimidin-4-one; 1-Phenyl-5,6-di-p-tolyl-1,5-dihydro-pyrazolo[3,4-d]pyrimidin-4-one; 6-(4-Chloro-phenyl)-5-(4-ethoxy-phenyl)-1-phenyl-1,5-dihydro-pyrazolo[3,4-d]pyrimidin-4-one; 5-(4-Fluoro-phenyl)-1-phenyl-6-p-tolyl-1,5-dihydro-pyrazolo[3,4-d]pyrimidin-4-one; 5,6-Bis-(4-bromo-phenyl)-1-phenyl-1,5-dihydro-pyrazolo[3,4-d]pyrimidin-4-one; 5,6-Bis-(4-chloro-phenyl)-1-phenyl-1,5-dihydro-pyrazolo[3,4-d]pyrimidin-4-one; 5-(3-Chloro-phenyl)-6-(2-fluoro-phenyl)-1-phenyl-1,5-dihydro-pyrazolo[3,4-d]pyrimidin-4-one; 6-(2-Fluoro-phenyl)-1,5-diphenyl-1,5-dihydro-pyrazolo[3,4-d]pyrimidin-4-one; 1,5-Diphenyl-6-p-tolyl-1,5-dihydro-pyrazolo[3,4-d]pyrimidin-4-one; 5-(3-Chloro-phenyl)-6-(4-chloro-phenyl)-1-phenyl-1,5-dihydro-pyrazolo[3,4-d]pyrimidin-4-one; 5-(4-Methoxy-phenyl)-1,6-diphenyl-1,5-dihydro-pyrazolo[3,4-d]pyrimidin-4-one; 5-(3-Chloro-phenyl)-1-phenyl-6-p-tolyl-1,5-dihydro-pyrazolo[3,4-d]pyrimidin-4-one; 1-Phenyl-5,6-di-o-tolyl-1,5-dihydro-pyrazolo[3,4-d]pyrimidin-4-one; 6-(4-Chloro-phenyl)-1-phenyl-5-p-tolyl-1,5-dihydro-pyrazolo[3,4-d]pyrimidin-4-one; 6-(4-Bromo-phenyl)-5-(2,4-dimethyl-phenyl)-1-phenyl-1,5-dihydro-pyrazolo[3,4-d]pyrimidin-4-one; 6-(4-Chloro-phenyl)-1-phenyl-5-m-tolyl-1,5-dihydro-pyrazolo[3,4-d]pyrimidin-4-one; 6-(4-Bromo-phenyl)-1-phenyl-5-o-tolyl-1,5-dihydro-pyrazolo[3,4-d]pyrimidin-4-one; 6-(2-Fluoro-phenyl)-1-phenyl-5-o-tolyl-1,5-dihydro-pyrazolo[3,4-d]pyrimidin-4-one; 5-(4-Isopropyl-phenyl)-1-phenyl-6-m-tolyl-1,5-dihydro-pyrazolo[3,4-d]pyrimidin-4-one; 6-(4-Bromo-phenyl)-1-phenyl-5-m-tolyl-1,5-dihydro-pyrazolo[3,4-d]pyrimidin-4-one; 6-(4-Bromo-phenyl)-5-(4-ethoxy-phenyl)-1-phenyl-1,5-dihydro-pyrazolo[3,4-d]pyrimidin-4-one; 5-(4-Chloro-phenyl)-6-(2-fluoro-phenyl)-1-phenyl-1,5-dihydro-pyrazolo[3,4-d]pyrimidin-4-one; 5-(3-Chloro-phenyl)-1-phenyl-6-o-tolyl-1,5-dihydro-pyrazolo[3,4-d]pyrimidin-4-one; 5-(3,5-Dimethyl-phenyl)-1-phenyl-6-m-tolyl-1,5-dihydro-pyrazolo[3,4-d]pyrimidin-4-one; 6-(4-Bromo-phenyl)-5-(4-fluoro-phenyl)-1-phenyl-1,5-dihydro-pyrazolo[3,4-d]pyrimidin-4-one; 5-(4-Fluoro-phenyl)-1-phenyl-6-o-tolyl-1,5-dihydro-pyrazolo[3,4-d]pyrimidin-4-one; 1-Phenyl-5-m-tolyl-6-p-tolyl-1,5-dihydro-pyrazolo[3,4-d]pyrimidin-4-one; 5-(4-Bromo-phenyl)-1,6-diphenyl-1,5-dihydro-pyrazolo[3,4-d]pyrimidin-4-one; 5-(3-Chloro-phenyl)-1,6-diphenyl-1,5-dihydro-pyrazolo[3,4-d]pyrimidin-4-one; 1,6-Diphenyl-5-m-tolyl-1,5-dihydro-pyrazolo[3,4-d]pyrimidin-4-one; 5-(4-Ethoxy-phenyl)-1,6-diphenyl-1,5-dihydro-pyrazolo[3,4-d]pyrimidin-4-one; 6-(4-Bromo-phenyl)-5-(3-chloro-phenyl)-1-phenyl-1,5-dihydro-pyrazolo[3,4-d]pyrimidin-4-one; 6-(4-Chloro-phenyl)-1-phenyl-5-o-tolyl-1,5-dihydro-pyrazolo[3,4-d]pyrimidin-4-one; 6-(4-Chloro-phenyl)-5-(3,5-dimethyl-phenyl)-1-phenyl-1,5-dihydro-pyrazolo[3,4-d]pyrimidin-4-one; 1-Phenyl-6-o-tolyl-5-p-tolyl-1,5-dihydro-pyrazolo[3,4-d]pyrimidin-4-one; 1,5,6-Triphenyl-1,5-dihydro-pyrazolo[3,4-d]pyrimidin-4-one; 5-(4-Bromo-phenyl)-6-(4-chloro-phenyl)-1-phenyl-1,5-dihydro-pyrazolo[3,4-d]pyrimidin-4-one; 6-(2-Fluoro-phenyl)-5-(4-isopropyl-phenyl)-1-phenyl-1,5-dihydro-pyrazolo[3,4-d]pyrimidin-4-one; 6-(2-Fluoro-phenyl)-1-phenyl-5-p-tolyl-1,5-dihydro-pyrazolo[3,4-d]pyrimidin-4-one; 6-(4-Bromo-phenyl)-5-(4-isopropyl-phenyl)-1-phenyl-1,5-dihydro-pyrazolo[3,4-d]pyrimidin-4-one; 6-(4-Bromo-phenyl)-5-(4-chloro-phenyl)-1-phenyl-1,5-dihydro-pyrazolo[3,4-d]pyrimidin-4-one; 5-(4-Fluoro-phenyl)-1-phenyl-6-m-tolyl-1,5-dihydro-pyrazolo[3,4-d]pyrimidin-4-one; 1,6-Diphenyl-5-o-tolyl-1,5-dihydro-pyrazolo[3,4-d]pyrimidin-4-one; 5-(4-Ethoxy-phenyl)-6-(2-fluoro-phenyl)-1-phenyl-1,5-dihydro-pyrazolo[3,4-d]pyrimidin-4-one; and 1,6-Diphenyl-5-p-tolyl-1,5-dihydro-pyrazolo[3,4-d]pyrimidin-4-one.

Anti-obesity compounds, including Xenical®, Meridia® and cannabinoid receptor antagonists.

Inhibitors of platelet aggregation include Plavix®, aspirin and Clopidgrel®.

The structure of the active agents identified by generic or tradenames may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

Any person skilled in the art is fully enabled to identify the active agents and, based on these references, likewise enabled to manufacture and test the pharmaceutical indications and properties in standard test models, both in vitro and in vivo.

Another aspect of the present invention relates to methods for the prevention, delay of progression or treatment of conditions mediated by the PPAR receptor activity in mammals such as a diabetic disease or disorder, hyperlipidemic disease or disorder, a metabolic disease or disorder, a cardiovascular disease or disorder or an addictive disease or disorder comprising administration of a therapeutically effective amount of a PPAR agonist, or pharmaceutically acceptable salts thereof, alone or in combination with at least one active ingredient selected from the group consisting of
(i) HDL increasing compounds;
(ii) anti-diabetics;
(iii) an anti-hypertensive agent;
(iv) cholesterol absorption modulator;
(v) apo-A1 analogs and mimetics;
(vi) renin inhibitors;
(vii) thrombin inhibitors;
(viii) aldosterone inhibitors;
(ix) GLP-1 agonists;
(x) glucagon receptor antagonists;
(xi) cannabinoid receptor 1 antagonists;
(xii) anti-obesity agents; and
(xiii) inhibitors of platelet aggregation
or, in each case, a pharmaceutically acceptable salt thereof;
   and optionally a pharmaceutically acceptable carrier to a warm-blooded mammal in need thereof. Such conditions include addictive disorders such as nicotine addiction, cocaine addiction and the like, dyslipidemia, hyperlipidemia, hypercholesteremia, atherosclerosis, hypertriglyceridemia, heart failure, myocardial infarction, vascular diseases, cardiovascular diseases, stroke, intermittent claudication, restenosis after PCTA, hypertension, obesity including reduction in CV risk in obese patients, inflammation, arthritis, cancer including breast, colon and prostate cancer, Alzheimer's disease, skin disorders, respiratory diseases, ophthalmic disorders, IBDs (irritable bowel disease), Crohn's disease, hypofibrinolysis, hypercoaguable state, metabolic/cardiometabolic syndrome, elevated CRP, appearance of microalbuminuria, reduction of proteinuria, renal failure (DM, non-DM), NASH (non alcoholic steato hepatitis) non-alcoholic fatty liver, CV events in patients with high CRP, vascular dementia, psoriasis, ischaemia reperfusion injury, asthma, COPD, eosinophilia, RA, airway hyperresponsiveness (AHR), inflammatory digestive diseases (e.g. ulcerative colitis) diseases of antigen-induced inflammatory responses. The compound(s) of the present invention are particularly useful in mammals as hypoglycemic agents for the treatment and prevention of conditions such as impaired glucose tolerance, hyperglycemia, insulin resistance, type-1 and type-2 diabetes and Syndrome X. Also contemplated is the administration of the compound(s) of the present invention for the improvement of cardiac metabolism and cardioprotection in heart transplant patients.

In another aspect the pharmaceutical compositions of the present invention may be used to facilitate smoking cessation, temporary abstinence or smoking reduction and therefore prevention, delay of progression or treatment of conditions associated with smoking such as craving for nicotine and the increased appetite, dysphoria or depressed mood, sleeplessness, irritability, frustration, anger, anxiety, difficulty in concentrating and restlessness.

The pharmaceutical activities as effected by administration of the pharmaceutically active agent(s) according to the present invention can be demonstrated e.g. by using corresponding pharmacological models known in the pertinent art. The person skilled in the pertinent art is fully enabled to select a relevant animal test model to prove the hereinbefore and hereinafter indicated therapeutic indications and beneficial effects.

Protocols demonstrating tests for determining the activity of a compound or combination of compounds of the present invention with respect to smoking cessation, are disclosed in Paterson, N. et al., Psychopharmacology, 167:257-264, 2003, Kenny, P. J. et al, Ann. N.Y. Acad. Sci., 1003: 415-418 (2003), WO2004002463, WO0237927, WO0158450 in paragraph [0049]; WO9511679, Example III, and Example IV; WO0043002 Example 1, 2, 3 and 4; WO9733581 Example 1, 2, 3, 4, and 5; and WO9917803, all of which are expressly incorporated herein in their entireties by reference.

Illustrative of the invention administration of at 5 mg/kg/day in ob/ob mice for seven days led Compared to vehicle, administration of (R)-1-{4-[5-methyl-2-(4-trifluoromethyl-phenyl)-oxazol-4-ylmethoxy]-benzenesulfonyl}-2,3-dihydro-1H-indole-2-carboxylic acid treatment at 5 mg/kg/day significantly lowered food intake starting on day 3, and by day 7 the daily food intake was down by 50% (p<0.01) in ob/ob mice. Also, Chrna2 (cholinergic receptor, neuronal nicotinic, alpha polypeptide 2) was the most upregulated gene in the duodenum, expression being increased by 15, 11 and almost 140 fold (p<0.01) respectively, after 1, 2 and 7 days of treatment with (R)-1-{4-[5-methyl-2-(4-trifluoromethyl-phenyl)-oxazol-4-ylmethoxy]-benzenesulfonyl}-2,3-dihydro-1H-indole-2-carboxylic acid.

A “diabetic disease or disorder” as defined in this application comprises, but is not limited to hyperglycemia, hyperinsulinaemia, diabetes, insulin resistance, impaired glucose metabolism, conditions of impaired glucose tolerance (IGT), conditions of impaired fasting plasma glucose, obesity, diabetic retinopathy, diabetic nephropathy, glomerulosclerosis, diabetic neuropathy and syndrome X.

A “hyperlipidemic disease or disorder” as defined in this application comprises, but is not limited to hyperlipidaemia, hypertriglyceridemia, coronary heart disease, vascular restenosis, endothelial dysfunction, obesity and impaired vascular compliance.

A “metabolic disease or disorder” as defined in this application comprises, but is not limited to obesity.

A “cardiovascular disease or disorder” as defined in this application comprises, but is not limited to hypertension, congestive heart failure, diabetes, glomerulosclerosis, chronic renal failure, coronary heart disease, angina pectoris, myocardial infarction, stroke, vascular restenosis endothelial dysfunction, impaired vascular compliance and congestive heart failure.

Hypertension, especially in connection with a “cardiovascular disease or condition”, includes and is not limited to mild, moderate and severe hypertension as defined in Journal of Hypertension 1999, 17:151-183, especially on page 162. Especially preferred is “isolated systolic hypertension” (ISH).

Preferably, the therapeutically effective amounts of the active agents according to the invention can be administered simultaneously or sequentially in any order, e.g. separately or in a fixed combination.

In the case of the combinations of active agents of the present invention, all the more surprising is that the combined administration of (a) a PPAR agonist or pharmaceutically acceptable salts thereof and

(b) at least one active ingredient selected from the group consisting of

(i) HDL increasing compounds;
(ii) anti-diabetics;
(iii) an anti-hypertensive agent;
(iv) cholesterol absorption modulator;
(v) apo-A1 analogs and mimetics;
(vi) renin inhibitors;
(vii) thrombin inhibitors;
(viii) aldosterone inhibitors;
(ix) GLP-1 agonists;
(x) glucagon receptor antagonists;
(xi) cannabinoid receptor 1 antagonists;
(xii) anti-obesity agents; and
(xiii) inhibitors of platelet aggregation
or, in each case, a pharmaceutically acceptable salt thereof;
   and optionally a pharmaceutically acceptable carrier, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions.

The term “potentiation” shall mean an increase of a corresponding pharmacological activity or therapeutical effect, respectively. Potentiation of one component of the combination according to the present invention by co-administration of an other component according to the present invention means that an effect is being achieved that is greater than that achieved with one component alone or that is greater than the sum of effects of each component.

The term “synergistic” shall mean that the drugs, when taken together, produce a total joint effect that is greater than the sum of the effects of each drug when taken alone.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

With respect to the combinations according to the present invention as described hereinbefore and hereinafter they may be used for simultaneous use or sequential use in any order, e.g. for separate use or as a fixed combination.

The combinations according to the present invention comprises a “kit of parts” in the sense that the components can be dosed independently or by use of different fixed combinations with distinguished amounts of the components at different time points. The parts of the “kit of parts” can then e.g. be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components. Preferably, there is at least one beneficial effect, e.g. a mutual enhancing of the effect of a pharmaceutical combination comprising (a) a PPAR agonist or pharmaceutically acceptable salts thereof and (b) at least one active ingredient selected from the group consisting of
(i) HDL increasing compounds;
(ii) anti-diabetics;
(iii) an anti-hypertensive agent;
(iv) cholesterol absorption modulator;
(v) apo-A1 analogs and mimetics;
(vi) renin inhibitors;
(vii) thrombin inhibitors;
(viii) aldosterone inhibitors;
(ix) GLP-1 agonists;
(x) glucagon receptor antagonists;
(xi) cannabinoid receptor 1 antagonists;
(xii) anti-obesity agents; and
(xiii) inhibitors of platelet aggregation
or, in each case, a pharmaceutically acceptable salt thereof;
   and optionally a pharmaceutically acceptable carrier, in particular a potentiation or a synergism, e.g. a more than additive effect, additional advantageous effects, less side effects, a combined therapeutical effect in a non-effective dosage of one or each of the components, especially a potentiation or synergism.

The invention furthermore relates to a commercial package comprising the pharmaceutical active compounds according to the present invention together with instructions for simultaneous, separate or sequential use.

These pharmaceutical preparations are for oral administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. These are prepared in a manner that is known per se, for example using conventional mixing, granulation, coating, solubulizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound(s) can depend on a variety of factors, such as mode of administration, homeothermic species, age and/or individual condition.

Preferred dosages for the active ingredients of the pharmaceutically active compound(s) according to the present invention are therapeutically effective dosages, especially those that are commerically available.

Normally, in the case of oral administration of pharmaceutical composition in accordance with the present invention, an approximate daily dose of from about 1 mg to about 360 mg is to be estimated, preferably a daily dose of from 1 mg to 100 mg, more preferably a daily dose of from 1 mg to 50 mg, e.g. for a patient of approximately 75 kg in weight.

In case of ACE inhibitors, preferred dosage unit forms of ACE inhibitors are, for example, tablets or capsules comprising e.g. from about 5 mg to about 20 mg, preferably 5 mg, 10 mg, 20 mg or 40 mg, of benazepril; from about 6.5 mg to 100 mg, preferably 6.25 mg, 12.5 mg, 25 mg, 50 mg, 75 mg or 100 mg, of captopril; from about 2.5 mg to about 20 mg, preferably 2.5 mg, 5 mg, 10 mg or 20 mg, of enalapril; from about 10 mg to about 20 mg, preferably 10 mg or 20 mg, of fosinopril; from about 2.5 mg to about 4 mg, preferably 2 mg or 4 mg, of perindopril; from about 5 mg to about 20 mg, preferably 5 mg, 10 mg or 20 mg, of quinapril; or from about 1.25 mg to about 5 mg, preferably 1.25 mg, 2.5 mg, or 5 mg, of ramipril. Preferred is t.i.d. administration.

Although the present invention has been described in considerable detail with reference to certain preferred versions thereof, other versions are possible without departing from the spirit and scope of the preferred versions contained herein. All references and patents (U.S. and others) referred to herein are hereby incorporated by reference in their entirety as if set forth in full herein.

We claim:

1. A pharmaceutical composition, comprising a PPAR agonist, or a pharmaceutically acceptable salt thereof, alone or in combination with at least one active ingredient selected from the group consisting of
(i) HDL increasing compounds;
(ii) anti-diabetics;
(iii) an anti-hypertensive agent;
(iv) cholesterol absorption modulator;
(v) apo-A1 analogs and mimetics;
(vi) renin inhibitors;
(vii) thrombin inhibitors;
(viii) aldosterone inhibitors;
(ix) GLP-1 agonists;
(x) glucagon receptor antagonists;
(xi) cannabinoid receptor 1 antagonists;
(xii) anti-obesity agents; and
(xiii) inhibitors of platelet aggregation or, in each case, a pharmaceutically acceptable salt thereof and a pharmaceutically acceptable carrier.

2. The pharmaceutical composition according to claim 1 wherein the PPAR agonist is a PPAR alpha agonist.

3. The pharmaceutical composition of claim 1 wherein the PPAR agonist is a dual PPAR alpha/gamma agonist.

4. The pharmaceutical composition of claim 3 wherein the dual PPAR alpha/gamma agonist is of formula

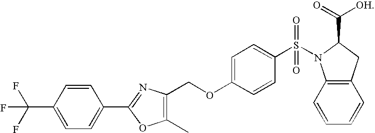

5. The pharmaceutical composition of claim 3 wherein the dual PPAR alpha/gamma agonist is 3-isobutyl-8-(6-methoxy-isoquinolin-4-ylmethyl)-1-methyl-3,7-dihydro-purine-2,6-dione.

6. The pharmaceutical composition of claim 1 wherein the anti-diabetic agent is selected from the group consisting of insulin sensitivity enhancers and non-glitazone type PPAR gamma agonists.

7. The pharmaceutical composition of claim 1 wherein the anti-hypertensive agents are selected from the group consisting of ACE inhibitors, renin inhibitors, calcium channel blockers, diuretics, beta-blockers and AT′ receptor antagonists.

8. The pharmaceutical composition of claim 1 wherein the cholesterol absorption modulators is ezetimibe.

9. The pharmaceutical composition of claim 1 wherein the oral thrombin inhibitor is ximelagatrap.

10. The pharmaceutical composition of claim 1 wherein the inhibitor of platelet aggregation is clopidogrel.

11. A method for the treatment of a diabetic disease or disorder, a hyperlipidemic disease or disorder, a metabolic disease or disorder and/or a cardiovascular disease or disorder or an addictive disease or disorder, comprising:
administration of a therapeutically effective amount of a PPAR agonist, or a pharmaceutically acceptable salt thereof, alone or in combination with at least one active ingredient selected from the group consisting of
(i) HDL increasing compounds;
(ii) anti-diabetics;
(iii) an anti-hypertensive agent;
(iv) cholesterol absorption modulator;
(v) apo-A1 analogs and mimetics;
(vi) renin inhibitors;
(vii) thrombin inhibitors;
(viii) aldosterone inhibitors;
(ix) GLP-1 agonists;
(x) glucagon receptor antagonists;
(xi) cannabinoid receptor 1 antagonists;
(xii) anti-obesity agents; and
(xiii) inhibitors of platelet aggregation
or, in each case, a pharmaceutically acceptable salt thereof and a pharmaceutically acceptable carrier to a warm-blooded mammal in need thereof

12. The method of claim 11 wherein the disease or disorder is selected from nicotine addiction, cocaine addiction, dyslipidemia, hyperlipidemia, hypercholesteremia, atherosclerosis, hypertriglyceridemia, heart failure, myocardial infarction, vascular diseases, cardiovascular diseases, stroke, intermittent claudication, restenosis after PCTA, hypertension, obesity including reduction in CV risk in obese patients, inflammation, arthritis, cancer including breast, colon and prostate cancer, Alzheimer's disease, skin disorders, respiratory diseases, ophthalmic disorders, IBDs (irritable bowel disease), Crohn's disease, hypofibrinolysis, hypercoaguable state, metabolic/cardiometabolic syndrome, elevated CRP, appearance of microalbuminuria, reduction of proteinuria, renal failure (DM, non-DM), NASH (non alcoholic steato hepatitis) non-alcoholic fatty liver, CV events in patients with high CRP, vascular dementia, psoriasis, ischaemia reperfusion injury, asthma, CORD, eosinophilia, RA, airway hyperresponsiveness (AHR), inflammatory digestive diseases, diseases of antigen-induced inflammatory responses, impaired glucose tolerance, hyperglycemia, insulin resistance, type-1 and type-2 diabetes and Syndrome X.

13. The method of claim 11 for the improvement of cardiac metabolism and cardioprotection in heart transplant patients.

14. The method of claim 11 for facilitating smoking cessation, temporary abstinence or smoking reduction.

15. The method of claim 11 for treatment of conditions associated with smoking.

16. The method of claim 15 wherein the conditions associated with smoking are craving for nicotine and the increased appetite, dysphoria or depressed mood, sleeplessness, irritability, frustration, anger, anxiety, difficulty in concentrating and restlessness smoking cessation or reduction.
